(19) United States
(12) Patent Application Publication
(10) Pub. No.: US 2014/0363871 A1
(43) Pub. Date: Dec. 11, 2014

(54) PROCESS FOR OBTAINING COPPER NANOPARTICLES FROM A FUNGUS SELECTED BETWEEN HYPOCREA LIXII AND TRICHODERMA KONINGIOPSIS AND USE OF FUNGI SELECTED BETWEEN HYPOCREA LIXII AND TRICHODERMA KONINGIOPSIS IN BIOREMEDIATION OF WASTEWATER AND PRODUCTION OF COPPER NANOPARTICLES

(71) Applicants: VALE S.A., Rio de Janeiro (BR); UNIVERSIDADE DE SÃO PAULO - USP, Sao Paulo (BR)

(72) Inventors: Benedito CORRÊA, Sao Paulo (BR); Cláudio Augusto Oller NASCIMENTO, Sao Paulo (BR); Márcia Regina SALVADORI, Salto (BR)

(21) Appl. No.: 14/297,417

(22) Filed: Jun. 5, 2014

Related U.S. Application Data
(60) Provisional application No. 61/831,362, filed on Jun. 5, 2013.

(51) Int. Cl. C12P 3/00; B22F 9/00; C02F 1/28

(52) U.S. Cl. CPC C12P 3/00; C02F 1/286; B22F 9/00; C02F 2101/20
USPC 435/168; 210/660; 420/469

(57) ABSTRACT

The present invention refers to a process for obtaining copper nanoparticles from a fungus selected between Hypocrea lixii and Trichoderma koningiopsis.

The present invention refers to the use of dead biomass of Hypocrea lixii or Trichoderma koningiopsis to perform bioremediation of wastewater and for industrial scale production of copper nanoparticles.

In the present invention, it is developed a synthetic strategy for the biosynthesis and removal of copper nanoparticles which is fast, low cost, environment friendly and easily scalable, using as a reduction agent the fungus Hypocrea lixii or Trichoderma koningiopsis.

### FIELD OF THE INVENTION

The present invention refers to a process for obtaining copper nanoparticles from a fungus selected between Hypocrea lixii and Trichoderma koningiopsis.

The present invention refers to the use of dead biomass of fungus selected between Hypocrea lixii and Trichoderma koningiopsis, to perform bioremediation of copper-containing wastewater, in order to produce copper nanoparticles. The invention allows producing copper nanoparticles in industrial scale.

### BACKGROUND OF THE INVENTION

Heavy metals are the major contaminants in rivers and industrial effluents. To be very reactive and bioaccumulative element in living organisms, heavy metals have received special attention, since some are extremely toxic even in very low amounts, for instance chromium, cadmium and mercury. The use of fungi and yeasts in the removal or reduction of these pollutants is an environmentally suitable alternative, since the environmental impact caused by these types of remediation is small.

Recently, synthesis of inorganic nanoparticles has been demonstrated by many physical and chemical means. But the importance of biological synthesis is being emphasized globally at present because chemical methods are capital intensive toxic, non-ecofriendly and have low productive [Varshney R, Bhadauria S, Gaur M S (2012) A review: Biological synthesis of silver and copper nanoparticles. Nano Biomed Eng 4: 99-106]. Copper nanoparticles, due to their unique physical and chemical properties and the low cost of preparation, have been of great interest recently. Furthermore, copper nanoparticles have potential industrial use such as gas sensors, catalytic processes, high temperature superconductors, solar cells, wood preservative treatment and so on [Li Y, Liang J, Tao Z, Chen J (2007) CuO particles and plates: Synthesis and gas-sensor application. Mater Res Bull 43: 2380-2385; and Guo Z, Liang X, Pereira T, Scaffaro R, Hahn H T (2007) CuO nanoparticle filled vinyl-ester resin nanocomposites: Fabrication, characterization and property analysis. Compos Sci Tech 67: 2036-2044].

New alternatives for the synthesis of metallic nanoparticles are currently being explored through bacteria, fungi, yeast and plants [Thakkar K N, Mhatre S S, Parikh R Y (2010) Biological synthesis of metallic nanoparticles. Nanomedicine 6: 257-262]. Wastewater from copper mining often contain a high concentration of this toxic metal generated during the extraction, beneficiation, and processing of metal. In recent years, the bioremediation, through of the biosorption of toxic metals as copper has received a great deal of attention not only as a scientific novelty, but also because of its potential industrial applications.

This novel approach is competitive, effective, and cheap [Volesky B (2001) Detoxification of metal bearing effluents: biosorption for the next century. Hydrometallurgy 59: 203-216]. In this respect, fungi have been used in bioremediation processes since they are a versatile group that can adapt to and grow under various extreme conditions of pH, temperature and nutrient availability, as well as at high concentrations of metals [Anand P, Isar j, Saran S, Saxena R K (2006) Bioaccumulation of copper by Trichoderma viride. Bioresource Technol 97: 1018-1025]. Consequently, there has been considerable interest in developing biosynthesis methods for the preparation of copper nanoparticles as an alternative to physical and chemical methods.

Literature review of previous studies revealed that few articles were published on biosynthesis of copper nanoparticles [Varshney R, Bhadauria S, Gaur M S (2012) A review: Biological synthesis of silver and copper nanoparticles. Nano Biomed Eng 4: 99-106] and none of the studies used the fungi Hypocrea lixii (H. lixii) and Trichoderma koningiopsis (T. koningiopsis). Also, most of the biosynthesis studies on copper nanoparticles focused on biorreduction phase only and ignored the important biosorption phase of the process.

Studying towards the goal to enlarge the scope of biological systems for the biosynthesis of metallic nanomaterials and bioremediation of wastewater, it is explored for the first time the use of the fungi H. lixii and T. koningiopsis, to the uptake and reduction of copper ions to copper nanoparticles. Thus, the bioremediation and green synthesis of copper nanoparticles, has been achieved in the present study using dead biomass of H. lixii and T. koningiopsis.

### BRIEF DESCRIPTION OF THE FIGURES

FIG. 1 shows Batch biosorption studies. Influence of the physico-chemical factors on the live, dried and dead biomass of H. lixii. (A) Effect of the amount of biosorbent. (B) Effect of pH. (C) Effect of temperature. (D) Effect of contact time. (E) Effect of agitation rate. (F) Effect of initial copper concentration.

FIG. 2 shows Biosorption isotherm models and biosorption kinetics of H. lixii. Langmuir plots for live (A), dried (B) and dead (C) biomass. Pseudo second-order models for live (D), dried (E) and dead (F) biomass.

FIG. 3 shows TEM micrographs of H. lixii sections. (A) Control (without copper), (B) Section of the fungus showing extracellular localization of copper nanoparticles and (C) Copper nanoparticles.

FIG. 4 shows Dead biomass of H. lixii analyzed by SEM-EDS. (A) Control (without copper) and (B) biomass exposed to copper.

FIG. 5 shows EDS spectra recorded of dead biomass of H. lixii. (A) before exposure to copper solution and (B) after exposure to copper

FIG. 6 shows FTIR spectra of dead biomass of H. lixii. (A) before and (B) after to saturation with copper ions.

FIG. 7 shows Batch biosorption studies. Influence of the physico-chemical factors on the live, dried and dead biomass of T. koningiopsis. (A) Effect of the amount of biosorbent. (B) Effect of pH. (C) Effect of temperature. (D) Effect of contact time. (E) Effect of agitation rate. (F) Effect of initial copper concentration.

FIG. 8 shows Biosorption isotherm models and biosorption kinetics of T. koningiopsis. Langmuir plots for live (A), dried (B) and dead (C) biomass. Pseudo second-order models for live (D), dried (E) and dead (F) biomass.

FIG. 9 shows TEM micrographs of T. koningiopsis sections. (A) before contact with the metal ion showing the cell wall, cytoplasmic membrane and cytoplasm with no metal, (B) after contact with the metal ion copper showing the nanoparticles (darkest arrow) and its adhesion in the region outer cell wall (arrow lighter) and (C) aggregate of nanoparticles (darkest arrow) adhered to the outer region of the cell wall (arrow clearer).

FIG. 10 shows Dead biomass of T. koningiopsis analyzed by SEM-EDS. (A) Control (without copper) and (B) biomass exposed to copper.

FIG. 11 shows EDS spectra recorded of dead biomass of T. koningiopsis. (A) before exposure to copper solution and (B) after exposure to copper

FIG. 12 shows FTIR spectra of dead biomass of T. koningiopsis (A) before and (B) after to saturation with copper ions.

### SUMMARY OF THE INVENTION

The present invention refers to a process for obtaining copper nanoparticles from a fungus selected between H. lixii and T. koningiopsis.

The present invention refers also to the use of dead biomass of H. lixii to perform bioremediation of wastewater and for industrial scale production of copper nanoparticles.

Further, the present invention also refers to the use of dead biomass of T. koningiopsis to perform bioremediation of wastewater and for industrial scale production of copper nanoparticles.

### DETAILED DESCRIPTION OF THE INVENTION

A biological system for the biosynthesis of nanoparticles and uptake of copper from wastewater using dead biomass of H. lixii and T. koningiopsis was analyzed and described for the first time.

In the present invention, it is explored for the first time the extracellular biosynthesis and uptake of copper nanoparticles from wastewater utilizing the dead biomass of the filamentous fungi T. koningiopsis and H. lixii.

In the present invention, it is developed a synthetic strategy for the biosynthesis and removal of copper nanoparticles which is fast, low cost, environment friendly and easily scalable, using as a reduction agent the fungi T. koningiopsis and H. lixii.

The present invention refers to a process for obtaining copper nanoparticles from a fungus selected between H. lixii and T. koningiopsis comprising the following steps:
a. Isolation of a fungus selected from H. lixii and T. koningiopsis;
b. Determination of copper tolerance of the isolated fungus of step a;
c. Preparation of a copper stock solution;
d. Addition of said isolated fungus in the medium culture Sabouraud broth resulting in a live biomass;
e. Subjecting the live biomass to autoclave resulting in a dead biomass;
f. Drying live biomass resulting in a dried biomass; and
g. Determination of copper nanoparticles retention in the live, dried and dead biomass

The determination of copper retention by biosorption of the isolated fungus is performed by addition for each one of the biomasses (live, dried and dead) in a copper solution item [0027] step c;

The biosorption of copper onto dead, dried and live biomass of fungus was performed in function of the: initial metal concentrations (50-500 mg L⁻¹), pH (2-6), temperature (20-60° C.), agitation (50-250 rpm), inoculum volume (0.15-1.0 g) and contact time (5-360 min).

The development of the invention will be illustrated by the following no-exhaustive examples.

### EXAMPLE

### Brief Summary of the Tests and Results

The equilibrium and kinetics investigation of the biosorption of copper onto dead, dried and live biomass of fungus was performed in function of the initial metal concentration, pH, temperature, agitation and inoculum volume.

The range of biosorption capacity of cooper was observed for dead biomass, completed within 60 min of contact, at pH 5.0, temperature of 40° C., at agitation speed of 150 rpm with a maximum biosorption of copper of 15-30 mg g⁻¹ for H. lixii and 20-35 mg g⁻¹ for T. koningiopsis.

The equilibrium data were better described using the Langmuir isotherm and Kinetic analysis indicated the pseudo-second-order model.

The average size, morphology and location of nanoparticles biosynthesized by the fungus were determined by scanning electron microscopy (SEM), energy dispersive X-ray spectroscopy (EDS) and transmission electron microscopy (TEM).

The shape of nanoparticles was found to be mainly spherical with an average size of 15-35 nm and synthesized extracellularly for H. lixii and 70-95 nm and rarely aggregates of 328.27 nm and were synthesized extracellularly for T. koningiopsis. Fourier transform infrared spectroscopy (FTIR) with Attenuated total reflectance (ATR) study disclosed that the amide groups were bound to the particles, which was accountable for the stability of nanoparticles. It further confirmed the presence of protein as the stabilizing and capping agent surrounding the copper nanoparticles.

These studies demonstrate that dead biomass of H. lixii and of T. koningiopsis offers an economical and technically feasible option for bioremediation of wastewater and for industrial scale production of copper nanoparticles.

### Example 1

### Use of Dead Biomass of Hypocrea lixii to Perform Bioremediation of Wastewater and for Industrial Scale Production of Copper Nanoparticles

### 1. Growth and Maintenance of the Organism

H. lixii was isolated from the water collected from a pond of copper waste from Sossego mine, located in Canãa dos Carajás, Pará, Brazilian Amazonia region (06° 26′S latitude and 50° 4′ W longitude). H. lixii was maintained and activated in Sabouraud Dextrose Agar (SDA) (Oxoid, England) [Kumar B N, Seshadri N, Ramana D K V, Seshaiah K, Reddy A V R (2011) Equilibrium, Thermodynamic and Kinetic studies on Trichoderma viride biomass as biosorbent for the removal of Cu (II) from water. Separ Sci Technol 46: 997-1004].

### 2. Minimum Inhibitory Concentration in Agar Medium

Copper tolerance of the isolated fungus was determined as the minimum inhibitory concentration (MIC) by the spot plate method [Ahmad I, Ansari M I, Aqil F (2006) Biosorption of Ni, Cr and Cd by metal tolerante Aspergillus niger and Penicillium sp using single and multi-metal solution. Indian J Exp Biol 44: 73-76]. SDA plates containing different concentrations of copper (50 to 2000 mg L⁻¹) were prepared and inocula of the tested fungus were spotted onto the metal and control plates (plate without metal). The plates were incubated at 25° C. for at least 5 days. The MIC is defined as the lowest concentration of metal that inhibits visible growth of the isolate.

### 3. Determination of Copper Nanoparticles Retention by the Biosorbent

### 3.1. Preparation of the Adsorbate Solutions

All chemicals used in the present study were of analytical grade and were used without further purification. All dilutions were prepared in double-deionized water (Milli-Q Millipore 18.2 Ωcm⁻¹ conductivity). The copper stock solution was prepared by dissolving CuCl₂.2H₂O (Carlo Erba, Italy) in double-deionized water. The working solutions were prepared by diluting this stock solution.

### 3.2. Biomass Preparation

The fungal biomass was prepared in the Sabouraud broth (Sb) (Oxoid, England), and incubated at 25° C. for 5 days, at 150 rpm. After incubation, the pellets were harvested and washed with of double-deionized water this was referred to as live biomass. For the preparation of dead biomass, an appropriate amount of live biomass was autoclaved. The dried biomass was obtained through drying of the fungal mat at 50° C. until it became crispy. The dried mat was ground to obtain uniform sized particles [Salvadori M R, Lepre L F, Ando R A, do Nascimento C A O, Corrêa B (2013) Biosynthesis and uptake of copper nanoparticles by dead biomass of Hypocrea lixii isolated from the metal mine in the Brazilian Amazon region. Plos One 8: 1-8].

### 3.3. Studies of the Effects of Physico-Chemical Factors on the Efficiency of Adsorption of Copper Nanoparticles by the Biosorbent

The pH (2-6), temperature (20-60° C.), contact time (5-360 min), initial copper concentration (50-500 mg L⁻¹), and agitation rate (50-250 rpm) on the removal of copper was analysed. Such experiments were optimized at the desired pH, temperature, metal concentrations, contact time, agitation rate and biosorbent dose (0.15-1.0 g) using 45 mL of 100 mg L⁻¹ of Cu (II) test solution in plastic flask.

Several concentrations (50-500 mg L⁻¹) of copper (II) were prepared by appropriate dilution of the copper (II) stock solution. The pH was adjusted with HCl or NaOH. The desired biomass dose was then added and the content of the flask was shaken for the desired contact time in an electrically thermostatic reciprocating shaker at the required agitation rate. After shaking, the Cu (II) solution was separated from the biomass by vacuum filtration through a Millipore membrane. The metal concentration in the filtrate was determined by flame atomic absorption spectrophotometer (AAS). The efficiency (R) of metal removal was calculated using following equation:

R=(Cᵢ−Cₑ)/Cᵢ·100

where Cᵢ and Cₑ are initial and equilibrium metal concentrations, respectively. The metal uptake capacity, qₑ, was calculated using the following equation:

qₑ=V(Cᵢ−Cₑ)/M

where qₑ (mg g⁻¹) is the biosorption capacity of the biosorbent at any time, M (g) is the biomass dose, and V (L) is the volume of the solution.

### 3.4. Biosorption Isotherm Models

Biosorption was analyzed by the batch equilibrium technique using the following sorbent concentrations of 50-500 mg L¹. The equilibrium data were fit using Freundlich and Langmuir isotherm models [Volesky B (2003) Biosorption process simulation tools. Hydrometallurgy 71: 179-190]. The linearized Langmuir isotherm model is:

Cₑ/qₑ=1/(qₘ·b)+Cₑ/qₘ

where qₘ is the monolayer sorption capacity of the sorbent (mg g⁻¹), and b is the Langmuir sorption constant (L mg⁻¹). The linearized Freundlich isotherm model is:

ln qₑ=ln K_{F}+1/n·ln Cₑ

where K_{F} is a constant relating the biosorption capacity and 1/n is related to the adsorption intensity of adsorbent.

### 3.5. Biosorption Kinetics

The results of rate kinetics of Cu (II) biosorption were analyzed using pseudo-first-order, and pseudo-second-order models. The linear pseudo-first-order model [Lagergren S (1898) About the theory of so called adsorption of soluble substances. Kung Sven Veten Hand 24: 1-39] can be represented by the following equation:

log(qₑ−qₜ)=log qₑ−K₁/2.303·t

where, qₑ (mg g⁻¹) and qₜ (mg g⁻¹) are the amounts of adsorbed metal on the sorbent at the equilibrium time and at any time t, respectively, and K₁ (min⁻¹) is the rate constant of the pseudo-first-order adsorption process. The linear pseudo-second-order model [Ho Y S, Mckay G (1999) Pseudo-second-order model for sorption process. Process Biochem 34: 451-465] can be represented by the following equation:

t/qₜ=1/K₂·qₑ²+t/qₑ

where K₂ (g mg⁻¹ min⁻¹) is the equilibrium rate constant of pseudo-second-order.

### 4. Biosynthesis of Metallic Copper Nanoparticles by H. lixii

In this study was used only the dead biomass of H. lixii that showed a high adsorption capacity of copper metal ion compared to live and dried biomass. Biosynthesis of copper nanoparticles by dead biomass of H. lixii was investigated using the data of the equilibrium model at a concentration of 100 mg L⁻¹ of copper (II) solution.

### 4.1. TEM Observation

Analysis by Transmission electron microscopy (TEM) was used for determining the size, shape and location of copper nanoparticles on biosorbent, where cut ultra-thin of the specimens, were observed in a transmission electron microscope (JEOL-1010).

### 4.2. SEM-EDS Analysis

Analysis of small fragments of the biological material before and after the formation of copper nanoparticles, was performed on pin stubs and then coated with gold under vacuum and were examined by SEM on a JEOL 6460 LV equipped with an energy dispersive spectrometer (EDS).

### 4.3. FTIR-ATR Analysis

Infrared vibrational spectroscopy (FTIR) was used to identify the functional groups present in the biomass and to evaluate the spectral variations caused by the presence of copper nanoparticles. The infrared absorption spectra were obtained on Bruker model ALPHA interferometric spectrometer. The samples were placed directly into the sample compartment using an attenuated total reflectance accessory of single reflection (ATR with Platinium-crystal diamond). Eighty spectra were accumulated for each sample, using spectral resolution of 4 cm⁻¹.

H. lixii, isolated from copper mine, was subjected to minimum inhibitory concentration (MIC) at different copper concentrations (50-2000 mg L⁻¹) and the results indicated that H. lixii exhibited high tolerance to copper (528 mg L⁻¹).

### 4.4. Influence of the Physico-Chemical Factors on Biosorption

The present investigation showed that copper removal by H. lixii biomass was influenced by physico-chemical factors such as biomass dosage, pH, temperature, contact time, rate of agitation and metal ion concentration. The biosorbent dose is an important parameter since it determines the capacity of a biosorbent for a given initial concentration of the metals.

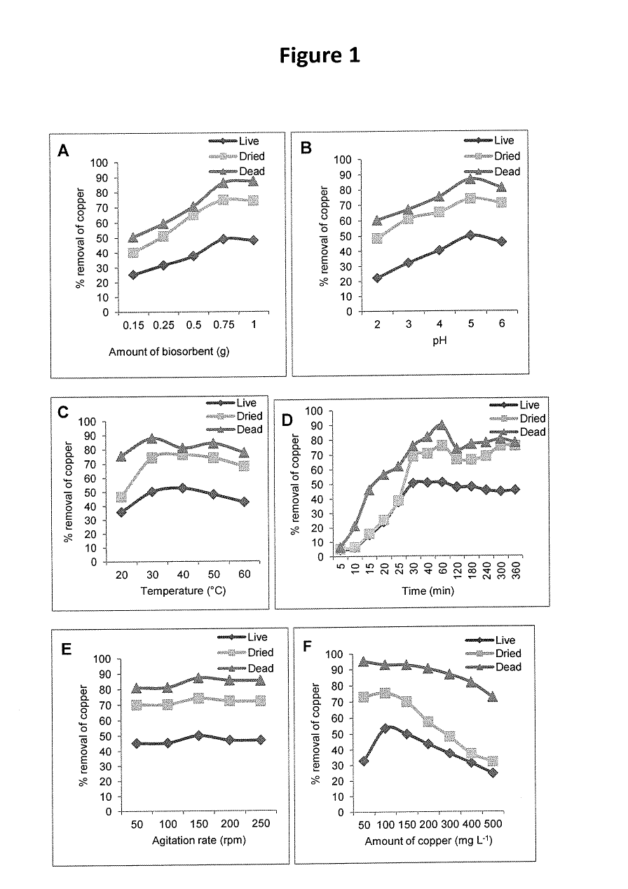

As observed in FIG. 1A, the removal of copper by live and dried biomass of H. lixii increased with increasing biomass concentration and reached saturation at 0.75 g L⁻¹, whereas the saturation was reached to 1.0 g L⁻¹ for dead biomass (FIG. 1A). The percent removal of copper by dead biomass was greater than that observed for live and dried biomass (FIG. 1A). The dead biomass for Cu (II) removal offers the following advantages: the metal removal system is not subjected to toxicity, it does not require growth media and adsorbed metal ions can be easily desorbed and dead biomass can be reutilized. Copper removal by live and dried biomass decreased with an increase of biomass concentration beyond 0.75 g L⁻¹.

This finding indicates that dead biomass possess a higher affinity for copper than live and dried biomass. The increase in removal capacity with increasing biomass dose can be attributed to a greater total surface area and a consequent larger number of binding sites. Maximum removal of copper was observed at pH 5.0 for the three types of biomass as shown in FIG. 1B. At lower pH value, the cell wall of H. lixii becomes positively charged and it is responsible for reduction in biosorption capacity. In contrast, at higher pH (pH 5), the cell wall surface becomes more negatively charged and therefore the biosorption of Cu (II) onto H. lixii is high due to attraction between the biomass and the positively charged metal ion.

The maximum removal of copper was observed at 40° C. for the three types of biomass (FIG. 1C). The effect of the temperature on biosorption of the metal suggested an interaction between the metal and the ligands on the cell wall. It is observed that the graph (FIG. 1D) follows the sigmoid kinetics which is characteristic of enzyme catalysis reaction for all the three types of biomass. The kinetics of copper nanoparticles formation to dead biomass showed that more than 89% of the particles were formed within the 60 min of the reaction, which suggests that the formation of copper nanoparticles is exponential. The optimum copper removal was observed at an agitation speed of 150 rpm for all three types of biomass (FIG. 1E). At high agitation speeds, vortex phenomena occur and the suspension is no longer homogenous, a fact impairing metal removal [Liu Y G, Fan T, Zeng G M, Li X, Tong Q, et al. (2006) Removal of cadmium and zinc ions from aqueous solution by living Aspergillus niger. Trans Nonferrous Met Soc China 16: 681-686].

The percentage of copper adsorption decreased with increasing metal concentration (50-500 mg L⁻¹) at the three types of biomass as shown in FIG. 1F.

### 4.5. Sorption Isotherm and Kinetics Models

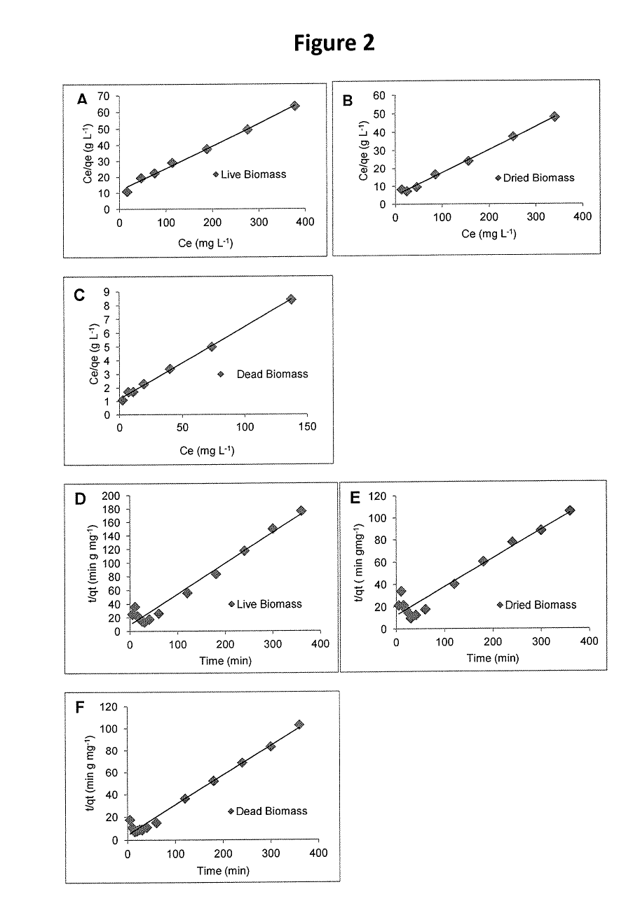

The Langmuir and Freundlich isotherm models were used to fit the biosorption data and to determine biosorption capacity. The Langmuir isotherm for Cu (II) biosorption obtained of the three types of H. lixii biomass is shown in FIG. 2A, FIG. 2B and FIG. 2C. The isotherm constants, maximum loading capacity estimated by the Langmuir and Freundlich models, and regression coefficients are shown in Table 1. The Langmuir model better described the Cu (II) biosorption isotherms than the Freundlich model.

The maximum adsorption rate of Cu (II) by H. lixii (19.0 mg g⁻¹) observed in this study was similar or higher than the adsorption rates reported for other known biosorbents, such as Pleurotus pulmonaris, Schizophyllum commune, Penicillium spp and Rhizopus arrhizus, with adsorption rates of 6.2, 1.52, 15.08 and 19.0 mg g⁻¹ respectively [Veit M T, Tavares C R G, Gomes-da-Costa S M, Guedes T A (2005) Adsorption isotherms of copper (II) for two species of dead fungi biomasses. Process Biochem 40: 3303-3308; Du A, Cao L, Zhang R, Pan R (2009) Effects of a copper-resistant fungus on copper adsorption and chemical forms in soils. Water Air Soil Poll 201: 99-107; Rome L, Gadd D M (1987) Copper adsorption by Rhizopus arrhizus, Cladosporium resinae and Penicillium italicum. Appl Microbiol Biotechnol 26: 84-90].

The kinetics of Cu (II) biosorption onto all three types of biomass of H. lixii were analysed using pseudo-first-order and pseudo-second-order models. All the constants and regression coefficients are shown in Table 2. In the present study, biosorption by H. lixii was best described using a pseudo-second-order kinetic model as shown in FIG. 2D, FIG. 2E and FIG. 2F. This adsorption kinetics is typical for the adsorption of divalent metals onto biosorbents [Reddad Z, Gerent C, Andres Y, LeCloirec P (2002) Adsorption of several metal ions onto a low-cost biosorbents: kinetic and equilibrium studies. Environ Sci Technol 36: 2067-2073].

### 4.6. Biosynthesis of Copper Nanoparticles

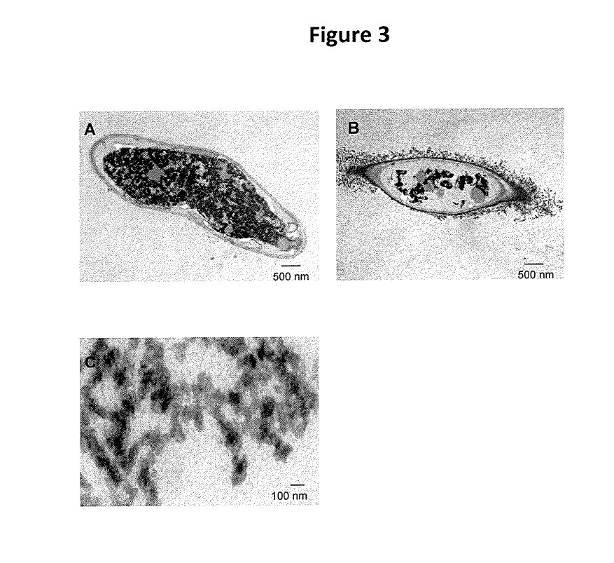

The studying of the involved mechanisms of the nanoparticles formation by biological systems is important in order to determine even more reliable and reproducible methods for its biosynthesis. To understanding the formation of nanoparticles in fungal biomass, was examined by TEM a fraction of the dead biomass. The location of the nanoparticles in H. lixii was investigated and the electron micrograph revealed that nanoparticles were found in the cell wall, but not in cytoplasm and cytoplasmic membrane, and was absent in control, the ultrastructural change such as shrinking of cytoplasmatic material was observed in control and biomass impregnated with copper due to autoclaving process (FIG. 3A and FIG. 3B). The extracellular location, offers the advantages of obtaining nanoparticles faster and in large amounts, easy removal and possible reuse of the biomass in the production process. The shape and size of nanoparticles are two of the most important features controlling the physical, chemical, optical and electronic properties of the nanoscopic materials [Alivisatos A P (1996) Perspectives on the physical chemistry of semiconductor nanocrystals. J Phys Chem 100: 13226-13239; Aizpurua J, Hanarp P, Sutherland D S, Käll M, Bryant G W, et al. (2003) Optical properties of gold nanorings. Phys Rev Lett 90: 57401-57404].

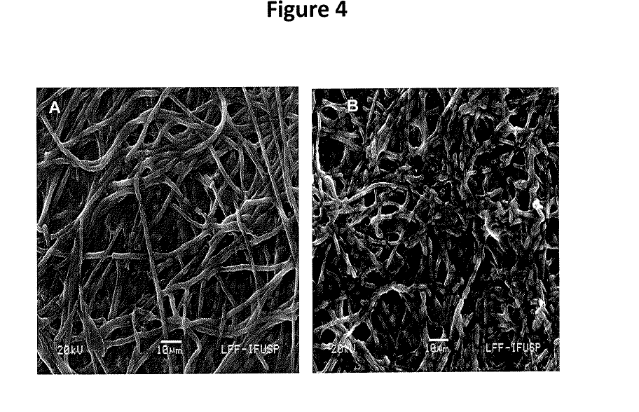

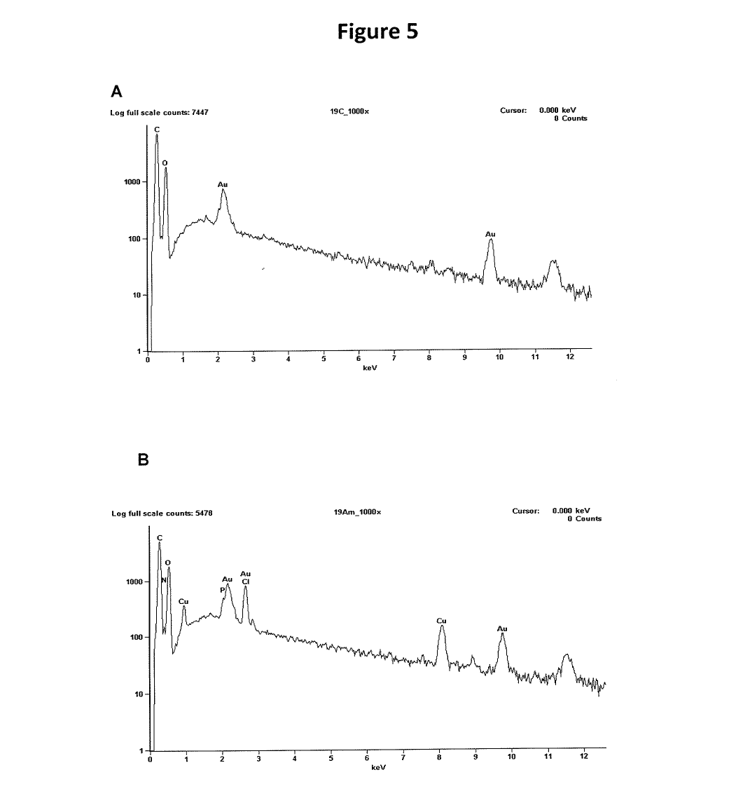

In this study copper nanoparticles showed an average diameter of 24.5 nm. At magnifications 100 nm, the particles are predominantly spherical as shown in FIG. 3C. The presence of copper nanoparticles was confirmed by spot profile SEM-EDS measurement. SEM micrographs recorded before and after biosorption of Cu (II) by fungal biomass was presented in FIG. 4A and FIG. 4B respectively. We observed that a surface modification occurred by increasing the irregularity, after binding of copper nanoparticles onto the surface of the fungus biomass. EDS spectra recorded in the examined region of the mycelium, show signals from copper (FIG. 5A and FIG. 5B) for the fungus.

Apart from this, the signals for C, N and 0 indicate the presence of proteins as a capping material on the surface of copper nanoparticles. Such signals are likely to be due to proteins secreted by the fungi, and is supported by FTIR-ATR measurement for the formation of copper nanoparticles, which identify the possible interactions between copper and bioactive molecules, which may be responsible for synthesis and stabilization (capping material) of copper nanoparticles.

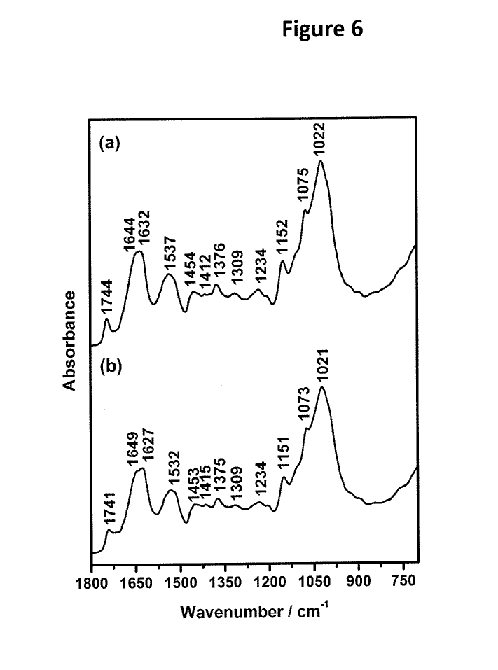

The amide linkages between amino acid residues in proteins give rise to well know signatures in the infrared region of the electro-magnetic spectrum. FTIR spectrum reveals two bands at 1649 and 1532 cm⁻¹, that correspond to the bending vibrations of amide I and amide II, respectively (FIG. 6). Such modes arise from peptides/proteins bound to copper nanoparticles, which suggests the possibility of these agents acting as capping agents [Bansal V, Ahamad A, Sastry M (2006) Fungus-mediated biotransformation of amorphous silica in rice husk to nanocrystalline Silica. J Am Chem Soc 128: 14059-14066].

In this study, after saturating the biomass samples with copper (II) ions, several bands shifts were observed in the FT-IR spectra in relation to pure samples, especially those assigned to amide groups. The bands at 1644, 1632 and 1537 cm⁻¹ were shifted to 1649, 1627 and 1532 cm⁻¹, respectively (FIG. 6). It suggests that biosorption is due to the interaction between copper ions and amide groups within the available biomass. The two bands observed at 1375 and 1073 cm⁻¹ can be assigned to the C-N stretching vibrations of the aromatic and aliphatic amines, respectively (FIG. 6) [Vigneshwaran N, Kathe A A, Varadarajan P V, Nachane R P, Balasubramanya R H (2007) Silver-protein (core-shell) nanoparticle production using spent mushroom substrate. Langmuir 23: 7113-7117].

Such observations indicate the presence and binding of proteins with copper nanoparticles which can lead to their possible stabilization. In dead biomass probably the protein from the cell is liberated during the autoclaving process and bound on the surface cell. This observation indicates that the copper nanoparticles in spherical morphology are present with proteins that are possibly bound to the surface of the nanoparticles thereby acting as stabilizing agents of the spherical nanoparticles. FTIR results obtained during the present study also revealed that amide groups from proteins have strong affinity to bind metals. However the type of protein involved in interactions with nanoparticles of copper which was studied remains to be determined. Such understanding may lead to a more efficient green process for the production of copper nanoparticles.

TABLE 1
Adsorption constants from simulations with Langmuir and Freundlich
models.

| Type of | Langmuir model | | | Freundlich model | | |
|---|---|---|---|---|---|---|
| biomass | qₘ (mg g⁻¹) | b (L mg⁻¹) | R² | K_{F} (mg g⁻¹) | 1/n | R² |
| Live | 7.2 | 0.012 | 0.993 | 0.44 | 0.44 | 0.972 |
| Dried | 8.0 | 0.025 | 0.995 | 0.59 | 0.39 | 0.857 |
| Dead | 19.0 | 0.044 | 0.997 | 1.37 | 0.51 | 0.966 |

TABLE 2
Kinetic parameters for adsorption of copper.

| | | |
|---|---|---|
| Type of | Pseudo-first-order | Pseudo-second-order |

| biomass | K₁ (min⁻¹) | R² | K₂ (g mg⁻¹ min⁻¹) | R² |
|---|---|---|---|---|
| Live | 2.30 × 10⁻³ | 0.026 | 19.78 × 10⁻³ | 0.968 |
| Dried | 1.51 × 10⁻² | 0.774 | 6.95 × 10⁻³ | 0.936 |
| Dead | 3.91 × 10⁻³ | 0.404 | 14.82 × 10⁻³ | 0.982 |

### Example 2

### Use of Dead Biomass of T. koningiopsis to Perform Bioremediation of Wastewater and for Industrial Scale Production of Copper Nanoparticles

### 1. Growth and Maintenance of the Organism

T. koningiopsis was isolated from the sediment collected from a pond of copper waste from Sossego mine, located in Cañaa dos Carajás, Para, Brazilian Amazonia region (06° 26′S latitude and 50° 4′ W longitude). T. koningiopsis was maintained and activated in Sabouraud Dextrose Agar (SDA) (Oxoid, England) [Kumar B N, Seshadri N, Ramana D K V, Seshaiah K, Reddy A V R (2011) Equilibrium, Thermodynamic and Kinetic studies on Trichoderma viride biomass as biosorbent for the removal of Cu (II) from water. Separ Sci Technol 46: 997-1004.].

### 2. Minimum Inhibitory Concentration in Agar Medium

Copper tolerance of the isolated fungus was determined as the minimum inhibitory concentration (MIC) by the spot plate method [Ahmad I, Ansari M I, Aqil F (2006) Biosorption of Ni, Cr and Cd by metal tolerante Aspergillus niger and Penicillium sp using single and multi-metal solution. Indian J Exp Biol 44: 73-76]. SDA plates containing different concentrations of copper (50 to 2000 mg L⁻¹) were prepared and inocula of the tested fungus were spotted onto the metal and control plates (plate without metal). The plates were incubated at 25° C. for at least 5 days. The MIC is defined as the lowest concentration of metal that inhibits visible growth of the isolate.

### 3. Determination of Copper Nanoparticles Retention by the Biosorbent

### 3.1. Preparation of the Adsorbate Solutions

All chemicals used in the present study were of analytical grade and were used without further purification. All dilutions were prepared in double-deionized water (Milli-Q Millipore 18.2 Ωcm⁻¹ conductivity). The copper stock solution was prepared by dissolving CuCl₂.2H₂O (Carlo Erba, Italy) in double-deionized water. The working solutions were prepared by diluting this stock solution.

### 3.2. Biomass Preparation

The fungal biomass was prepared in the Sabouraud broth (Sb) (Oxoid, England), and incubated at 25° C. for 5 days, at 150 rpm. After incubation, the pellets were harvested and washed with of double-deionized water this was referred to as live biomass. For the preparation of dead biomass, an appropriate amount of live biomass was autoclaved. The dried biomass was obtained through drying of the fungal mat at 50° C. until it became crispy. The dried mat was ground to obtain uniform sized particles [Salvadori M R, Lepre L F, Ando R A, do Nascimento C A O, Corrêa B (2013) Biosynthesis and uptake of copper nanoparticles by dead biomass of Hypocrea lixii isolated from the metal mine in the Brazilian Amazon region. Plos One 8: 1-8].

### 3.3. Studies of the Effects of Physico-Chemical Factors on the Efficiency of Adsorption of Copper Nanoparticles by the Biosorbent

The pH (2-6), temperature (20-60° C.), contact time (5-360 min), initial copper concentration (50-500 mg L⁻¹), and agitation rate (50-250 rpm) on the removal of copper was analysed. Such experiments were optimized at the desired pH, temperature, metal concentration, contact time, agitation rate and biosorbent dose (0.15-1.0 g) using 45 mL of 100 mg L⁻¹ of Cu (II) test solution in plastic flask.

Several concentrations (50-500 mg L⁻¹) of copper (II) were prepared by appropriate dilution of the copper (II) stock solution. The pH was adjusted with HCl or NaOH. The desired biomass dose was then added and the content of the flask was shaken for the desired contact time in an electrically thermostatic reciprocating shaker at the required agitation rate. After shaking, the Cu (II) solution was separated from the biomass by vacuum filtration through a Millipore membrane. The metal concentration in the filtrate was determined by flame atomic absorption spectrophotometer (AAS). The efficiency (R) of metal removal was calculated using following equation:

R=(Cᵢ−Cₑ)/Cᵢ·100

where Cᵢ and Cₑ are initial and equilibrium metal concentrations, respectively. The metal uptake capacity, qₑ, was calculated using the following equation:

qₑ=V(Cᵢ−Cₑ)/M

where qₑ (mg g⁻¹) is the biosorption capacity of the biosorbent at any time, M (g) is the biomass dose, and V (L) is the volume of the solution.

### 3.4. Biosorption Isotherm Models

Biosorption was analyzed by the batch equilibrium technique using the following sorbent concentrations of 50-500 mg L⁻¹. The equilibrium data were fit using Freundlich and Langmuir isotherm models [Volesky B (2003) Biosorption process simulation tools. Hydrometallurgy 71: 179-190]. The linearized Langmuir isotherm model is:

Cₑ/qₑ=1/(qₘ·b)+Cₑ/qₘ

where qₘ is the monolayer sorption capacity of the sorbent (mg g⁻¹), and b is the Langmuir sorption constant (L mg⁻¹). The linearized Freundlich isotherm model is:

ln gₑ=ln K_{F}+1/n·ln Cₑ

where K_{F} is a constant relating the biosorption capacity and 1/n is related to the adsorption intensity of adsorbent.

### 3.5. Biosorption Kinetics

The results of rate kinetics of Cu (II) biosorption were analyzed using pseudo-first-order, and pseudo-second-order models. The linear pseudo-first-order model [Lagergren S (1898) About the theory of so called adsorption of soluble substances. Kung Sven Veten Hand 24: 1-39] can be represented by the following equation:

log(qₑ−qₜ)=log qₑ−K₁/2.303·t

where, qₑ (mg g⁻¹) and qₜ (mg g⁻¹) are the amounts of adsorbed metal on the sorbent at the equilibrium time and at any time t, respectively, and K₁ (min⁻¹) is the rate constant of the pseudo-first-order adsorption process. The linear pseudo-second-order model [Ho Y S, Mckay G (1999) Pseudo-second-order model for sorption process. Process Biochem 34: 451-465] can be represented by the following equation:

t/qₜ=1/K₂·qₑ²+t/qₑ

where K₂ (g mg⁻¹ min⁻¹) is the equilibrium rate constant of pseudo-second-order.

### 4. Biosynthesis of Metallic Copper Nanoparticles by T. koningiopsis

In this study was used only the dead biomass of T. koningiopsis that showed a high adsorption capacity of copper metal ion compared to live and dried biomass. Biosynthesis of copper nanoparticles by dead biomass of T. koningiopsis was investigated using the data of the equilibrium model at a concentration of 100 mg L⁻¹ of copper (II) solution.

### 4.1. TEM Observation

Analysis by Transmission electron microscopy (TEM) was used for determining the size, shape and location of copper nanoparticles on biosorbent, where cut ultra-thin of the specimens, were observed in a transmission electron microscope (JEOL-1010).

### 4.2. SEM-EDS Analysis

Analysis of small fragments of the biological material before and after the formation of copper nanoparticles, was performed on pin stubs and then coated with gold under vacuum and were examined by SEM on a JEOL 6460 LV equipped with an energy dispersive spectrometer (EDS).

### 4.3. FTIR-ATR Analysis

Infrared vibrational spectroscopy (FTIR) was used to identify the functional groups present in the biomass and to evaluate the spectral variations caused by the presence of copper nanoparticles. The infrared absorption spectra were obtained on Bruker model ALPHA interferometric spectrometer. The samples were placed directly into the sample compartment using an attenuated total reflectance accessory of single reflection (ATR with Platinium-crystal diamond). Eighty spectra were accumulated for each sample, using spectral resolution of 4 cm⁻¹.

T. koningiopsis, isolated from copper mine, was subjected to minimum inhibitory concentration (MIC) at different copper concentrations (50-2000 mg L⁻¹) and the results indicated that T. koningiopsis exhibited high tolerance to copper (1057 mg L⁻¹).

### 4.4. Influence of the Physico-Chemical Factors on Biosorption

The present investigation showed that copper removal by T. koningiopsis biomass was influenced by physico-chemical factors such as biomass dosage, pH, temperature, contact time, rate of agitation and metal ion concentration.

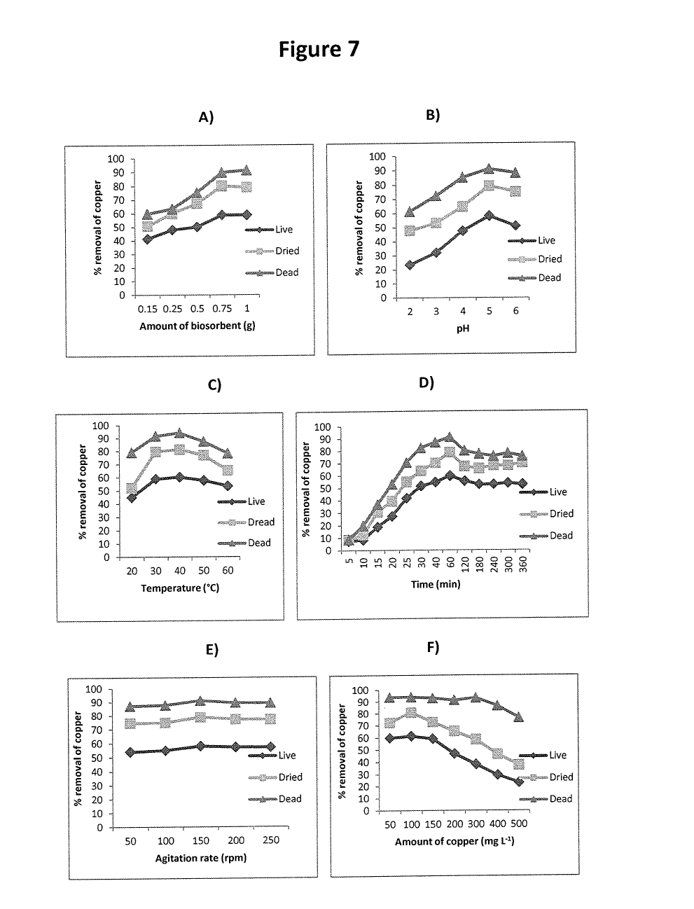

The biosorbent dose is an important parameter since it determines the capacity of a biosorbent for a given initial concentration of the metals. As observed in FIG. 7A, the removal of copper by live and dried biomass of T. koningiopsis increased with increasing biomass concentration and reached saturation at 0.75 g L⁻¹, whereas the saturation was reached to 1.0 g L⁻¹ for dead biomass (FIG. 7A).

The percent removal of copper by dead biomass was greater than that observed for live and dried biomass (FIG. 7A). The dead biomass for Cu (II) removal offers the following advantages: the metal removal system is not subjected to toxicity, it does not require growth media and adsorbed metal ions can be easily desorbed and dead biomass can be reutilized. Copper removal by live and dried biomass decreased with an increase of biomass concentration beyond 0.75 g L⁻¹.

This finding indicates that dead biomass possess a higher affinity for copper than live and dried biomass. The increase in removal capacity with increasing biomass dose can be attributed to a greater total surface area and a consequent larger number of binding sites. Maximum removal of copper was observed at pH 5.0 for the three types of biomass as shown in FIG. 7B. At lower pH value, the cell wall of T. koningiopsis becomes positively charged and it is responsible for reduction in biosorption capacity. In contrast, at higher pH (pH 5), the cell wall surface becomes more negatively charged and therefore the biosorption of Cu (II) onto T. koningiopsis is high due to attraction between the biomass and the positively charged metal ion.

The maximum removal of copper was observed at 40° C. for the three types of biomass (FIG. 7C). The effect of the temperature on biosorption of the metal suggested an interaction between the metal and the ligands on the cell wall. It is observed that the graph (FIG. 7D) follows the sigmoid kinetics which is characteristic of enzyme catalysis reaction for all the three types of biomass. The kinetics of copper nanoparticles formation to dead biomass showed that more than 91% of the particles were formed within the 60 min of the reaction, which suggests that the formation of copper nanoparticles is exponential. The optimum copper removal was observed at an agitation speed of 150 rpm for all three types of biomass (FIG. 7E). At high agitation speeds, vortex phenomena occur and the suspension is no longer homogenous, a fact impairing metal removal [Liu Y G, Fan T, Zeng G M, Li X, Tong Q, et al. (2006) Removal of cadmium and zinc ions from aqueous solution by living Aspergillus niger. Trans Nonferrous Met Soc China 16: 681-686].

The percentage of copper adsorption decreased with increasing metal concentration (50-500 mg L⁻¹) at the three types of biomass as shown in FIG. 7F.

### 4.5. Sorption Isotherm and Kinetics Models

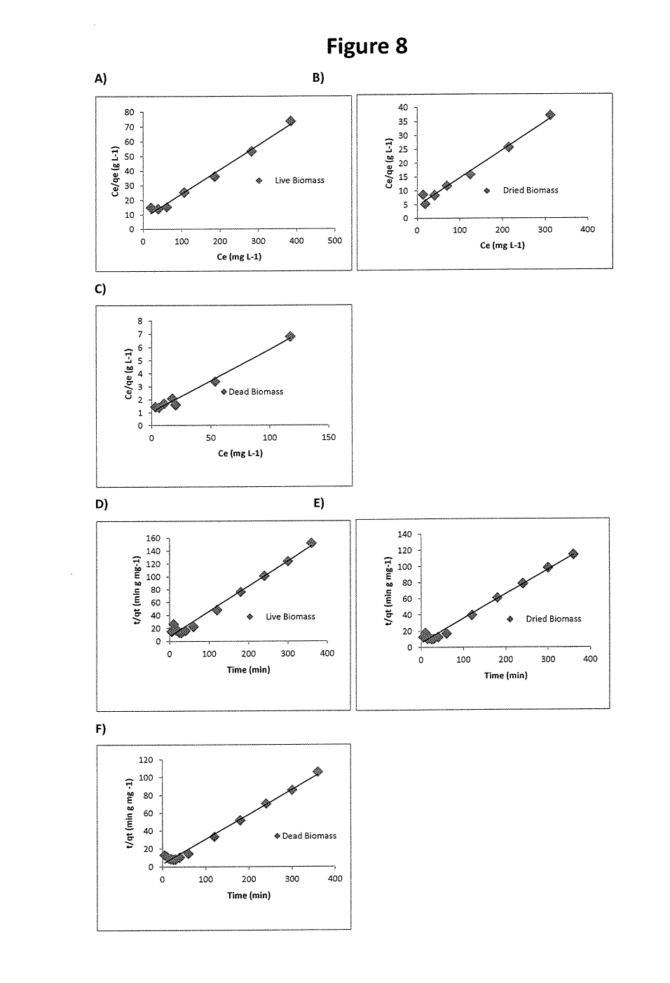

The Langmuir and Freundlich isotherm models were used to fit the biosorption data and to determine biosorption capacity. The Langmuir isotherm for Cu (II) biosorption obtained of the three types of T. koningiopsis biomass is shown in FIG. 8A, FIG. 8B and FIG. 8C. The isotherm constants, maximum loading capacity estimated by the Langmuir and Freundlich models, and regression coefficients are shown in Table 3. The Langmuir model better described the Cu (II) biosorption isotherms than the Freundlich model.

The maximum adsorption rate of Cu (II) by T. koningiopsis (21.1 mg g⁻¹) observed in this study was similar or higher than the adsorption rates reported for other known biosorbents, such as Pleurotus pulmonaris, Schizophyllum commune, Penicillium spp, Rhizopus arrhizus, Trichoderma viride, Pichia stipitis, Pycnoporus sanguineus with adsorption rates of 6.2, 1.52, 15.08, 19.0, 19.6, 15.85 and 2.76 mg g⁻¹ respectively [Veit M T, Tavares C R G, Gomes-da-Costa S M, Guedes T A (2005) Adsorption isotherms of copper (II) for two species of dead fungi biomasses. Process Biochem 40: 3303-3308; Du A, Cao L, Zhang R, Pan R (2009) Effects of a copper-resistant fungus on copper adsorption and chemical forms in soils. Water Air Soil Poll 201: 99-107; Rome L, Gadd D M (1987) Copper adsorption by Rhizopus arrhizus, Cladosporium resinae and Penicillium italicum. Appl Microbiol Biotechnol 26: 84-90; Kumar B N, Seshadri N, Ramana D K V, Seshaiah K, Reddy A V R (2011) Equilibrium, Thermodynamic and Kinetic studies on Trichoderma viride biomass as biosorbent for the removal of Cu (II) from water. Separ Sci Technol 46: 997-1004; Yilmazer P, Saracoglu N (2009) Bioaccumulation and biosorption of copper (II) and chromium (III) from aqueous solutions by Pichia stiptis yeast. J Chem Technol Biot 84: 604-610; Yahaya Y A, Matdom M, Bhatia S (2008) Biosorption of copper (II) onto immobilized cells of Pycnoporus sanguineus from aqueous solution: Equilibrium and Kinetic studies. J Hazard Mater 161: 189-195]

Comparison with biosorbents of bacterial origin showed that the Cu (II) adsorption rate of T. koningiopsis is comparable to that of Bacillus subtilis IAM 1026 (20.8 mg g⁻¹) [Nakajima A, Yasuda M, Yokoyama H, Ohya-Nishiguchi H, Kamada H (2001) Copper sorption by chemically treated Micrococcus luteus cells. World J Microb Biot 17: 343-347].

The kinetics of Cu (II) biosorption onto all three types of biomass of T. koningiopsis were analysed using pseudo-first-order and pseudo-second-order models. All the constants and regression coefficients are shown in Table 4. In the present study, biosorption by T. koningiopsis was best described using a pseudo-second-order kinetic model as shown in FIG. 8D, FIG. 8E and FIG. 8F. This adsorption kinetics is typical for the adsorption of divalent metals onto biosorbents [Reddad Z, Gerent C, Andres Y, LeCloirec P (2002) Adsorption of several metal ions onto a low-cost biosorbents: kinetic and equilibrium studies. Environ Sci Technol 36: 2067-2073].

### 4.6. Biosynthesis of Copper Nanoparticles

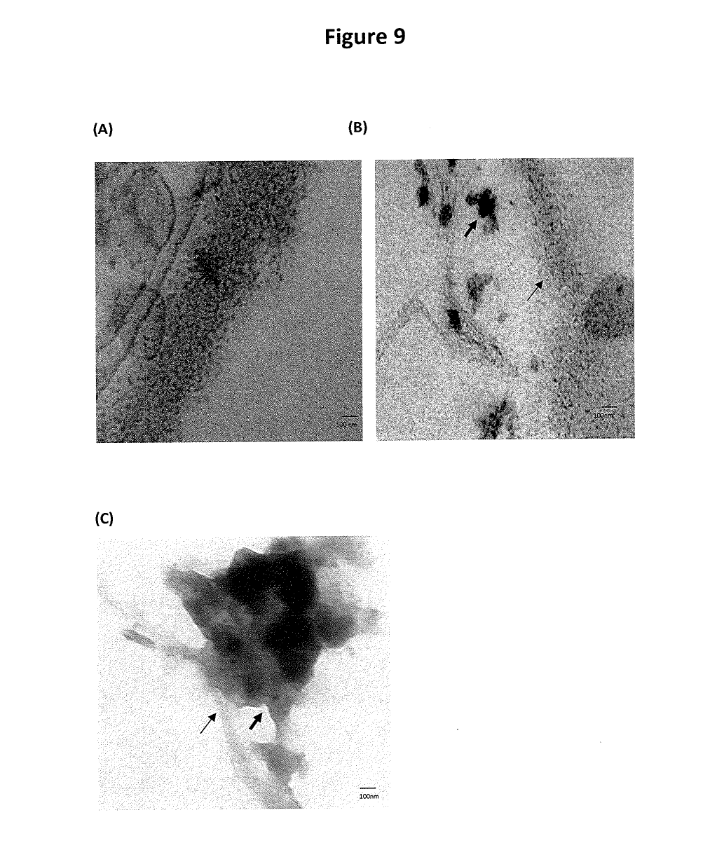

The studying of the involved mechanisms of the nanoparticles formation by biological systems is important in order to determine even more reliable and reproducible methods for its biosynthesis. To understanding the formation of nanoparticles in fungal biomass, was examined by TEM a fraction of the dead biomass. The location of the nanoparticles in T. koningiopsis was investigated and the electron micrograph revealed that nanoparticles were found in the cell wall, but not in cytoplasm and cytoplasmic membrane, and was absent in control, the ultrastructural change such as shrinking of cytoplasmatic material was observed in control and biomass impregnated with copper due to autoclaving process (FIG. 9A and FIG. 9B). The extracellular location, offers the advantages of obtaining nanoparticles faster and in large amounts, easy removal and possible reuse of the biomass in the production process. The shape and size of nanoparticles are two of the most important features controlling the physical, chemical, optical and electronic properties of the nanoscopic materials [Alivisatos A P (1996) Perspectives on the physical chemistry of semiconductor nanocrystals. J Phys Chem 100: 13226-13239; Aizpurua J, Hanarp P, Sutherland DS, Käll M, Bryant G W, et al. (2003) Optical properties of gold nanorings. Phys Rev Lett 90: 57401-57404].

In this study copper nanoparticles showed an average diameter of 87.5 nm and predominantly spherical as shown in FIG. 9B. Rare aggregates of nanoparticles were observed an average diameter of 328.27 nm (FIG. 9C).

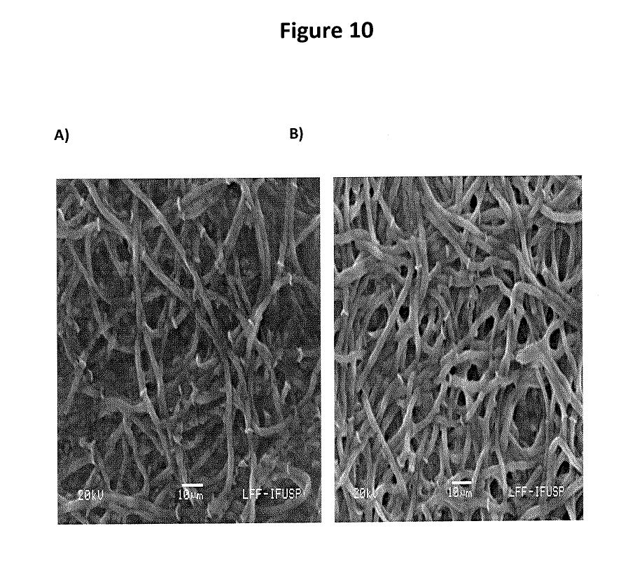

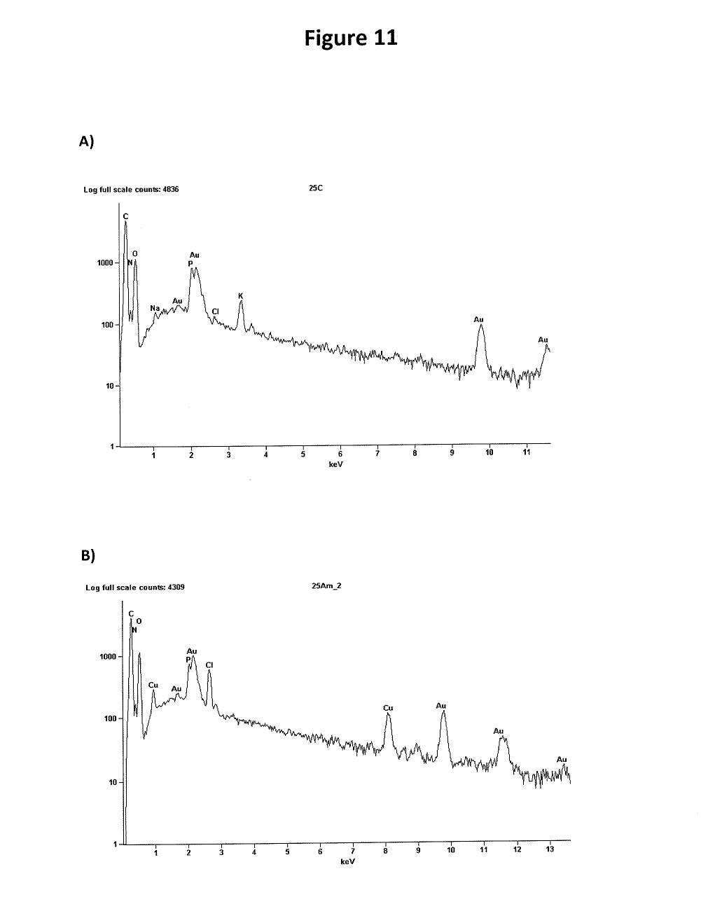

The presence of copper nanoparticles was confirmed by spot profile SEM-EDS measurement. SEM micrographs recorded before and after biosorption of Cu (II) by fungal biomass was presented in FIG. 10A and FIG. 10B respectively. We observed that a surface modification occurred by increasing the irregularity, after binding of copper nanoparticles onto the surface of the fungus biomass. EDS spectra recorded in the examined region of the mycelium, show signals from copper (FIG. 11A and FIG. 11B) for the fungus.

Apart from this, the signals for C, N, O, Na, P, Cl and K and indicate the presence of proteins as a capping material on the surface of copper nanoparticles. Such signals are likely to be due to proteins secreted by the fungi, and is supported by FTIR-ATR measurement for the formation of copper nanoparticles, which identify the possible interactions between copper and bioactive molecules, which may be responsible for synthesis and stabilization (capping material) of copper nanoparticles.

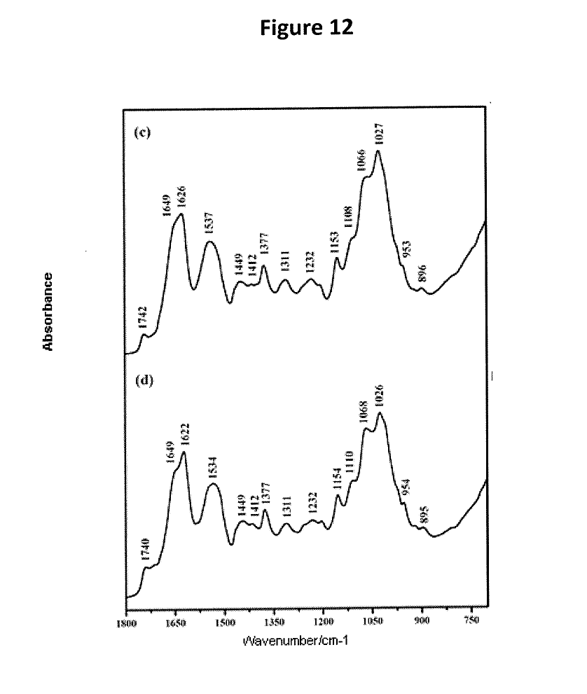

The amide linkages between amino acid residues in proteins give rise to well know signatures in the infrared region of the electro-magnetic spectrum. FTIR spectrum reveals two bands at 1649 and 1534 cm⁻¹, that correspond to the bending vibrations of amide I and amide II, respectively (FIG. 12). Such modes arise from peptides/proteins bound to copper nanoparticles, which suggests the possibility of these agents acting as capping agents [Bansal V, Ahamad A, Sastry M (2006) Fungus-mediated biotransformation of amorphous silica in rice husk to nanocrystalline Silica. J Am Chem Soc 128: 14059-14066].

In this study, after saturating the biomass samples with copper (II) ions, several bands shifts were observed in the FT-IR spectra in relation to pure samples, especially those assigned to amide groups. The bands at 1626 and 1537 cm⁻¹ were shifted to 1622 and 1534 cm⁻¹, respectively (FIG. 12). It suggests that biosorption is due to the interaction between copper ions and amide groups within the available biomass. The two bands observed at 1377 and 1068 cm⁻¹ can be assigned to the C-N stretching vibrations of the aromatic and aliphatic amines, respectively (FIG. 12) [Vigneshwaran N, Kathe A A, Varadarajan P V, Nachane R P, Balasubramanya R H (2007) Silver-protein (core-shell) nanoparticle production using spent mushroom substrate. Langmuir 23: 7113-7117].

Such observations indicate the presence and binding of proteins with copper nanoparticles which can lead to their possible stabilization. In dead biomass probably the protein from the cell is liberated during the autoclaving process and bound on the surface cell. This observation indicates that the copper nanoparticles in spherical morphology are present with proteins that are possibly bound to the surface of the nanoparticles thereby acting as stabilizing agents of the spherical nanoparticles. FTIR results obtained during the present study also revealed that amide groups from proteins have strong affinity to bind metals. However the type of protein involved in interactions with nanoparticles of copper which was studied remains to be determined. Such understanding may lead to a more efficient green process for the production of copper nanoparticles.

TABLE 3
Adsorption constants from simulations with Langmuir and
Freundlich models.

| Type of | Langmuir model | | | Freundlich model | | |
|---|---|---|---|---|---|---|
| biomass | qₘ(mg g⁻¹) | b (L mg⁻¹) | R² | K_{F} (mg g⁻¹) | 1/n | R² |
| Live | 6.0 | 0.021 | 0.989 | 0.41 | 0.41 | 0.815 |
| Dried | 10.0 | 0.021 | 0.984 | 0.44 | 0.47 | 0.817 |
| Dead | 21.1 | 0.043 | 0.984 | 1.10 | 0.57 | 0.8 98 |

TABLE 4
Kinetic parameters for adsorption of copper.

| | | |
|---|---|---|
| Type of | Pseudo-first-order | Pseudo-second-order |

| biomass | K₁ (min⁻¹) | R² | K₂ (g mg⁻¹ min⁻¹) | R² |
|---|---|---|---|---|
| Live | 4.83 × 10⁻³ | 0.395 | 18.90 × 10⁻³ | 0.981 |
| Dried | 4.37 × 10⁻³ | 0.469 | 15.26 × 10⁻³ | 0.986 |
| Dead | 2.53 × 10⁻³ | 0.139 | 19.35 × 10⁻³ | 0.987 |

1. PROCESS FOR OBTAINING COPPER NANOPARTICLES, from a fungus selected between Hypocrea lixii and Trichoderma koningiopsis comprising the following steps:
a. Isolation of a fungus selected from Hypocrea lixii and Trichoderma koningiopsis;
b. Determination of copper tolerance of the isolated fungus of step a;
c. Preparation of a copper stock solution;
d. Addition of said isolated fungus in the medium culture Sabouraud broth resulting in a live biomass;
e. Subjecting the live biomass to autoclave resulting in a dead biomass;
f. Drying live biomass resulting in a dried biomass; and
g. Determination of copper nanoparticles retention in the live, dried and dead biomass.

2. USE OF A FUNGUS, selected from Hypocrea lixii extract and Trichoderma koningiopsis extract to perform bioremediation of wastewater.

3. THE USE, according to claim 2, wherein Hypocrea lixii extract is dead mass of Hypocrea lixii.

4. THE USE, according to claim 2, wherein Trichoderma koningiopsis extract is dead mass of Trichoderma koningiopsis.

5. THE USE, according to one of the claims 1 to 4, wherein it is for the production of copper nanoparticles.

6. COPPER NANOPARTICLES, produced from a fungus selected between Hypocrea lixii and Trichoderma koningiopsis using during a bioremediation of wastewater.
